(19) United States
(12) Patent Application Publication
(10) Pub. No.: US 2020/0276089 A1
(43) Pub. Date: Sep. 3, 2020

(54) DENTAL GLASS POWDER AND DENTAL CEMENT

(71) Applicant: GC Corporation, Shizuoka (JP)

(72) Inventors: Ryosuke YOSHIMITSU, Tokyo (JP); Tomohiro HOSHINO, Tokyo (JP); Mizuki NAKAYAMA, Tokyo (JP)

(21) Appl. No.: 16/645,931

(22) PCT Filed: Aug. 16, 2018
(86) PCT No.: PCT/JP2018/030405

§ 371 Date: Mar. 10, 2020

(30) Foreign Application Priority Data
Oct. 2, 2017 (JP) 2017-192518

(51) Int. Cl. A61K 6/836; A61K 6/889

(52) U.S. Cl. CPC A61K 6/836; A61K 6/889

(57) ABSTRACT

An embodiment of the present invention, in a dental glass powder, contains zinc, silicon, fluorine and silver, and does not substantially contain aluminum.

### TECHNICAL FIELD

The present invention relates to a dental glass powder and dental cement.

### BACKGROUND ART

Aluminosilicate glass powder is commonly used as a dental glass powder.

Aluminosilicate glass powder is a glass powder that contains as its main ingredient oxides of Al (III) and Si (IV). Among aluminosilicate glass powders, fluoro-aluminosilicate glass powders are widely used as dental materials because the fluoro-aluminosilicate glass powders are expected to have a tooth reinforcement effect and a caries prevention effect due to fluorine (see, for example, Patent Documents 1 and 2).

Dental cement (glass ionomer cement) is known as the intended use of the fluoroaluminosilicate glass powder.

The glass ionomer cement is typically composed of a fluoroaluminosilicate glass powder, a polycarboxylic acid polymer, and a liquid containing water, wherein an acid-base reaction of the fluoroaluminosilicate glass powder and the polycarboxylic acid polymer results in the ionic cross-linking and the curing of the Al³⁺ eluted from the fluoroaluminosilicate glass powder and the conjugate base of the polycarboxylic acid polymer.

### PRIOR ART DOCUMENTS

### Patent Documents

Patent Document 1: Japanese Laid-Open Patent Application Publication No. 62-67008

Patent Document 2: Japanese Laid-Open Patent Application Publication No. 63-201038

### SUMMARY OF THE INVENTION

### Problem to be Solved by the Invention

However, it is demanded to improve a tooth demineralization inhibition effect and antimicrobial properties of dental cement.

Therefore, one embodiment of the present invention is intended to provide a dental glass powder capable of improving a tooth demineralization inhibition effect and antimicrobial properties of dental cement.

### Means for Solving the Problem

One aspect of the invention is a dental glass powder comprising zinc, silicon, fluorine and silver, and does not substantially contain aluminum.

### Effect of the Invention

According to an embodiment of the present invention, a dental glass powder capable of improving a tooth demineralization inhibition effect and antimicrobial properties of dental cement can be provided.

### EMBODIMENTS FOR IMPLEMENTING THE INVENTION

Next, an embodiment for carrying out the present invention will be described.

<Dental Glass Powder>

A dental glass powder of the present embodiment comprises zinc, silicon, fluorine and silver, and does not substantially contain aluminum. Therefore, it is possible to improve a tooth demineralization inhibition effect and antimicrobial properties of dental cement.

In the present specification and the claims, not substantially containing aluminum means that the aluminum content is not more than 1 mass % in terms of aluminum oxide (Al₂O₃).

This is based on the case where an aluminum compound is contained as impurities in the manufacturing process of the dental glass powder, even if the aluminum compound is not added to the raw material composition of the dental glass powder, and a detection error of the fluorescent X-ray analyzer that evaluates the composition of the dental glass powder. If the aluminum compound is not added to the raw material composition of the dental glass powder, the amount of aluminum contained in the dental glass powder is calculated in terms of aluminum oxide (Al₂O₃) and does not normally exceed 1 mass %.

The content of aluminum in the dental glass powder is preferably 0 to 0.5 mass %, and further preferably 0 to 0.3 mass %, which is calculated in terms of aluminum oxide (Al₂O₃).

The amount of zinc contained in the dental glass powder is preferably 10 to 60 mass % calculated in terms of zinc oxide (ZnO), more preferably 15 to 58 mass %, and further preferably 20 to 55 mass %. When the amount of zinc in the dental glass powder is 10 mass % or more calculated in terms of zinc oxide (ZnO), the dental cement decalcification inhibition effect can be improved, and when the amount is 60 mass % or less, the transparency of the dental glass powder can be improved.

The content of silicon in the dental glass powder is preferably 15 mass % to 50 mass %, and further preferably 20 mass % to 40 mass %, calculated in terms of silicon oxide (SiO₂). Here, silicon serves to form a network in glass. When the content of silicon in the dental glass powder is 15 mass % or more, which is calculated in terms of silicon oxide (SiO₂), the transparency of the dental glass powder can be improved, and when the content is 50 mass % or less, the curability of the dental cement can be improved.

The content of fluorine (F) in the dental glass powder is preferably in a range of 1 to 30 mass %, more preferably in a range of 2 to 20 mass %, and further preferably in a range of 3 to 10 mass %. If the content of fluorine (F) in the dental glass powder is 1 mass % or more, the dental glass powder can be expected to enhance the tooth, and if it is 30 mass % or less, it is possible to improve the curing of the dental cement.

The content of silver in the dental glass powder is preferably 1 to 15 mass %, more preferably 2 to 14 mass %, and further preferably 3 to 12 mass %, calculated as in terms of silver oxide (Ag₂O). When the content of silver in the dental glass powder is 1 mass % or more, which is calculated as in terms of silver oxide (Ag₂O), the antimicrobial properties of the dental glass powder can be improved, and when the content is 15 mass % or less, the transparency of the dental glass powder can be improved.

The dental glass powder may further contain calcium, phosphorus, strontium, lanthanum, sodium, potassium and the like.

The content of calcium in the dental glass powder is preferably 0 to 30 mass %, and further preferably 5 to 20 mass %, calculated in terms of calcium oxide (CaO). The inclusion of calcium in the dental glass powder can improve the operability of the dental cement.

The content of phosphorus in the dental glass powder is preferably 0 to 10 mass %, and further preferably 0 to 5 mass %, calculated in terms of phosphorus (V) oxide (P₂O₅). The inclusion of phosphorus in the dental glass powder can improve the operability of the dental cement.

The content of strontium in the dental glass powder is preferably 0 mass % to 40 mass %, and further preferably 10 mass % to 30 mass %, calculated in terms of strontium oxide (SrO). The inclusion of strontium in the dental glass powder can improve the radiopacity of the hardened dental cement.

The content of lanthanum in the dental glass powder is preferably 0 to 50 mass %, and further preferably 10 to 40 mass %, calculated in terms of lanthanum oxide (La₂O₃). The inclusion of lanthanum in the dental glass powder can improve the acid resistance of the hardened dental cement.

The content of sodium in the dental glass powder is preferably 0 to 15 mass %, and further preferably 1 to 10 mass %, calculated in terms of sodium oxide (Na₂O). The inclusion of sodium in the dental glass powder can improve the transparency of the dental glass powder.

The content of potassium contained in the dental glass powder is preferably 0 to 10 mass %, and further preferably 1 to 5 mass %, calculated in terms of potassium oxide (K₂O). The inclusion of potassium in the dental glass powder can improve the transparency of the dental glass powder.

The dental glass powder of the present embodiment can be applied to dental cement and the like.

<Method of Manufacturing Dental Glass Powder>

A dental glass powder according to the present embodiment can be produced by melting and pulverizing a raw material composition containing a zinc compound, a silicon compound, a fluorine compound, or a silver compound and not containing an aluminum compound.

The zinc compound is not particularly limited, but may be zinc oxide, zinc fluoride or the like, which may be used in combination with two or more kinds of compounds.

The silicon compound is not particularly limited, but may be anhydrous silicic acid or the like, which may be used in combination with two or more kinds of compounds.

The fluorine compound is not particularly limited, but may be calcium fluoride, strontium fluoride, sodium fluoride or the like, which may be used in combination with two or more kinds of compounds.

The silver compound is not particularly limited, but may be silver oxide, silver chloride, silver nitrate, silver sulfate, silver fluoride, silver bromide, silver iodide or the like, which may be used in combination with two or more kinds of compounds.

The raw composition may further include a calcium compound, a phosphorous compound, a strontium compound, a lanthanum compound, a sodium compound, a potassium compound and the like.

The calcium compound is not particularly limited, but may be calcium fluoride, calcium phosphate, calcium carbonate, calcium hydroxide or the like, which may be used in combination with two or more kinds of compounds.

The phosphorous compound is not particularly limited, but may be calcium phosphate, strontium phosphate, sodium dihydrogen phosphate or the like, which may be used in combination with two or more kinds of compounds.

The strontium compound is not particularly limited, but may be strontium fluoride, strontium hydroxide, strontium carbonate, strontium oxide, strontium phosphate or the like, which may be used in combination with two or more kinds of compounds.

The lanthanum compound is not particularly limited, but may be lanthanum fluoride, lanthanum oxide or the like, which may be used in combination with two or more kinds of compounds.

The sodium compound is not particularly limited, but may be sodium dihydrogen phosphate, sodium carbonate, sodium fluoride and the like, which may be used in combination with two or more kinds of compounds.

The potassium compound is not particularly limited, but may be potassium fluoride, potassium carbonate, potassium hydrogen carbonate, dipotassium hydrogen phosphate or the like, which may be used in combination with two or more kinds of compounds.

Each compound in the raw composition may be formulated so as to correspond to a composition other than aluminum in the dental glass powder.

The number average particle diameter of the dental glass powder according to the present embodiment is preferably 0.02 to 30 μm, and further preferably 0.02 to 20 μm. If the number average particle diameter of the dental glass powder is 0.02 μm or more, the operability of the dental cement can be improved, and if the number average particle diameter is 30 μm or less, the wear resistance of the hardened dental cement can be improved.

<Dental Cement>

The dental cement of the present embodiment has a first component containing a dental glass powder of the present embodiment and a second component containing a polycarboxylic acid-based polymer and water. Therefore, when the first component and the second component are mixed with each other, the conjugate base of the Zn²⁺ eluted from the dental glass powder and the polycarboxylic acid polymer is ionically cross-linked and cured by the acid-base reaction of the dental glass powder and the polycarboxylic acid polymer.

The polycarboxylic acid polymer is not limited, but may be a homopolymer or a copolymer of an α, β-unsaturated carboxylic acid.

The α, β-unsaturated carboxylic acid may be, for example, acrylic acid, methacrylic acid, 2-chloroacrylic acid, 3-chloroacrylic acid, aconitic acid, mesaconic acid, maleic acid, itaconic acid, fumaric acid, glutaconic acid, citraconic acid or the like.

Aldo, the polycarboxylic acid polymer may be a copolymer of the α, β-unsaturated carboxylic acid and a component capable of copolymerization with the α, β-unsaturated carboxylic acid.

The components that can copolymerize with the α, β-unsaturated carboxylic acids are, for example, acrylamide, acrylonitrile, methacrylate esters, acrylates, vinyl chloride, allyl chloride, vinyl acetate, and the like.

In this case, the ratio of the α, β-unsaturated carboxylic acid to the monomer constituting the polycarboxylic acid polymer is preferably not less than 50 mass %.

The polycarboxylic acid polymer is preferably a homopolymer or a copolymer of acrylic acid or itaconic acid.

Here, the first component may be either a powder component or a liquid component. Also, the second component is a liquid component.

The liquid component may be either a liquid or a paste.

Dental cement is used by mixing the first and second components and then kneading the admixture to produce a kneaded material of dental cement.

Here, at least part of the polycarboxylic acid polymer may be a powder.

Preferably, the mass ratio of the first component to the second component is 1 to 5 when preparing the admixture of dental cement. By setting the mass ratio of the first component to the second component at 1 or more, the strength of the hardened dental cement can be improved, and by setting the ratio at 5 or less, the operability of the dental cement can be improved.

### EXAMPLE

Hereinafter, working examples of the present invention will be described, but the present invention is not limited to the working examples.

### Working Examples 1 to 7

Zinc oxide (ZnO), anhydrous silicic acid (SiO₂), calcium fluoride (CaF₂), silver oxide (Ag₂O), calcium phosphate (Ca₃(PO₄)₂), strontium fluoride (SrF₂), phosphorus oxide (P₂O₅), lanthanum oxide (La₂O₃) and sodium fluoride (NaF) were mixed at a predetermined ratio, and then mixed and stirred sufficiently with a mortar, thereby producing a raw material composition. The raw material composition was placed in a platinum crucible and placed in an electric furnace. The electric furnace was heated to 1300° C., melted and homogenized sufficiently, and then circulated into water to form a bulk glass. The obtained bulk glass was pulverized with an alumina ball mill for 20 hours and then was caused to pass through a 120 mesh sieve to produce a glass powder.

### Comparative Examples 1 to 4

In preparing the raw material composition, a glass powder was produced in the same manner as the working examples 1 to 7, except that aluminum oxide (Al₂O₃) was added and was mixed in a predetermined proportion.

### Comparative Examples 5 and 6

In producing the raw material composition, a glass powder was produced in the same manner as the working examples 1 to 7, except that silver oxide (Ag₂O) was not added and was mixed in a predetermined proportion.

Next, the number average particle diameter and the composition of the glass powder were evaluated.

<Number Average Particle Diameter of Glass Powder>

The particle size distribution of the glass powder was measured using Laser Diffraction/Scattering Particle Size Distribution Analyzer LA-950 (manufactured by Horiba, Ltd.). The number average particle diameter of the glass powder in both the working examples and the comparative examples was 6 to 9 μm.

<Composition of Glass Powder>

The glass powder was analyzed and its composition was determined using ZSX Primus II (manufactured by Rigaku Corporation) of an X-ray fluorescence spectrometer.

Table 1 shows the evaluation results of the composition of the glass powder [mass %].

In the meantime, although the aluminum compound was not added to the glass powders of the working examples 1 to 7 and the comparative examples 5 and 6 when producing the raw material composition, 0.3 to 0.7 mass % of aluminum oxide (Al₂O₃) was detected. This is considered to be derived from the contamination of alumina from alumina balls or alumina pots used to pulverize the bulk glass, or the detection error of the X-ray fluorescence spectrometer.

Next, the dental decalcification inhibition effect and antimicrobial properties of the cement were evaluated.

<Production of Cement Admixture>

The glass powder as the first component and the 50 mass % aqueous solution of polyacrylic acid as the second component were mixed so that the mass ratio of the first component to the second component was 2.3 and then was kneaded to form kneaded cement.

<Inhibition Effect of Tooth Demineralization>

After bovine dentin was polished and planarized with water-resistant polished paper #1200 while supplying water, a polytetrafluoroethylene seal with a 3-mm diameter hole was attached to the polished surface of the bovine dentin. Next, after the kneaded cement was applied to a half of the surface of the hole in the polished surface of the bovine dentin on which the polytetrafluoroethylene seal was attached, the kneaded cement was hardened by leaving the bovine dentin in a thermostatic bath maintained at 37° C. and 100% relative humidity. The hardened bovine dentin was then immersed in a 37° C. demineralizing solution for 24 hours. At this time, the half of the surface of the hole in the polished surface of the bovine dentin on which the seal was attached was not hardened, and the surface that the demineralizing solution touches was made a test surface.

Here, the decalcifying liquid is a mixture of 50 mM aqueous acetic acid solution, 1.5 mM aqueous calcium chloride solution and 0.9 mM aqueous potassium dihydrogen phosphate solution, with a pH of 4.5.

The hardened bovine dentin was then cut and tested using a precise cutting machine to provide a thickness of 1 mm.

Next, the test specimen was photographed by a transmission method using an X-ray inspection apparatus, and the taken image was analyzed using image processing software to determine the amount of mineral loss, and the tooth demineralization inhibition effect was evaluated.

The criteria for the inhibition of tooth demineralization are as follows. The lower the mineral loss becomes, the higher the tooth demineralization inhibition effect becomes.

Good: Amount of mineral loss is less than 2300 volume %·μm

Poor: Amount of mineral loss is 2300 volume %·μm or more and 2800 volume %·μm or less

Very Poor: Mineral loss is 2800 volume %·μm or more

In the meantime, when the tooth demineralization inhibition effect of the produced test object was evaluated in the same manner as the above except that the kneaded cement was not applied, the mineral loss amount was not less than 4231 volume %·μm.

<Antimicrobial Properties>

After a mold having a diameter of 10 mm and a thickness of 2 mm was filled with the kneaded cement, the kneaded cement was hardened by leaving the kneaded cement in an environment of a temperature at 37° C. and 100% relative humidity for an hour. The hardened material was then removed from the mold and was immersed in 10 mL of BrainHart's Infusion (BHI) culture medium for 24 hours. Next, after removing the hardened material from the BHI culture medium, Streptococcus mutans (S. mutans) were seeded so that an OD600 value became 0.01, and were incubated at 37° C. for 24 hours. Next, the OD600 value of the BHI culture medium that cultured S. mutans was measured to assess its antimicrobial properties.

Here, the OD600 value means an optical density of 600 nm wavelength, and was measured using the plate reader SpectraMax M2 (manufactured by Molecular Devices Japan).

The determination criteria for antimicrobial properties are as follows. The lower the OD600 value, the higher the antimicrobial properties.

Good: If OD600 is less than 0.10

Poor: OD600 is not less than 0.10 and less than 0.20

Very Poor: OD600 is not less than 0.20

Table 1 shows the evaluation results of the dental decalcification inhibition effect and antimicrobial properties of the cement.

TABLE 1

| | WORKING EXAMPLE | | | | | | | COMPARATIVE EXAMPLE | | | | | |
|---|---|---|---|---|---|---|---|---|---|---|---|---|---|
| | 1 | 2 | 3 | 4 | 5 | 6 | 7 | 1 | 2 | 3 | 4 | 5 | 6 |
| SiO₂ | 29.7 | 30.5 | 28.6 | 26.7 | 34.8 | 32.1 | 37.2 | 27.2 | 23.6 | 25.4 | 25.5 | 31.4 | 33.0 |
| Al₂O₃ | 0.3 | 0.3 | 0.4 | 0.3 | 0.6 | 0.3 | 0.7 | 25.4 | 21.3 | 23.0 | 24.3 | 0.4 | 0.3 |
| F | 4.3 | 4.5 | 4.2 | 3.9 | 3.2 | 3.0 | 4.7 | 13.5 | 11.2 | 11.6 | 9.4 | 4.6 | 3.0 |
| CaO | 6.4 | 6.6 | 6.2 | 5.8 | | | 12.1 | | 1.8 | | | 6.8 | |
| SrO | | | | | 12.4 | 13.3 | | 15.8 | 28.0 | 22.3 | 30.2 | | 13.2 |
| La₂O₃ | 29.9 | 30.8 | 28.9 | 27.0 | | | | | 6.0 | | | 31.7 | |
| P₂O₅ | | | | | | 4.2 | | 12.1 | 3.5 | 10.6 | 4.4 | | |
| Na₂O | | | | | | | 3.5 | 6 | | | 5 | | |
| ZnO | 23.6 | 24.3 | 22.8 | 21.9 | 47.3 | 44.9 | 30.7 | | 4.6 | | | 25.1 | 50.5 |
| Ag₂O | 5.8 | 3.0 | 8.9 | 14.4 | 1.7 | 2.2 | 11.1 | | | 7.1 | 1.2 | | |
| DENTAL | GOOD | GOOD | GOOD | GOOD | GOOD | GOOD | GOOD | POOR | VERY | VERY | VERY | GOOD | GOOD |
| DECALCIFICATION | | | | | | | | | POOR | POOR | POOR | | |
| INHIBITION | | | | | | | | | | | | | |
| EFFECT | | | | | | | | | | | | | |
| MINERAL | 2046 | 2292 | 1912 | 2232 | 1736 | 1860 | 1653 | 2514 | 3015 | 3125 | 2924 | 2142 | 1553 |
| LOSS | | | | | | | | | | | | | |
| AMOUNT | | | | | | | | | | | | | |
| [volume % · μm] | | | | | | | | | | | | | |
| ANTIMICROBIAL | GOOD | GOOD | GOOD | GOOD | GOOD | GOOD | GOOD | VERY | VERY | GOOD | GOOD | POOR | POOR |
| PROPERTIES | | | | | | | | POOR | POOR | | | | |
| OD600 VALUE | 0.06 | 0.05 | 0.02 | 0.01 | 0.02 | 0.02 | 0.03 | 0.29 | 0.28 | 0.03 | 0.02 | 0.19 | 0.15 |

TABLE 1 indicates that the cement containing the glass powder of the working examples 1 to 7 has a high tooth demineralization effect and high antimicrobial properties.

In contrast, because the cement containing the glass powder of the comparative examples 1 to 4 has a content of aluminum oxide (Al₂O₃) in a range of 21.3 to 25.4 mass %, the tooth demineralization inhibition effect is low.

In addition, the cement containing the glass powder of the comparative examples 1, 2, 5 and 6 has low antimicrobial properties because the glass powder does not contain silver.

This international application claims priority to Japanese Patent Application No. 2017-192518, filed Oct. 2, 2017, and the entire contents of Japanese Patent Application No. 2017-192518 are incorporated herein by reference.

1. A dental glass powder, comprising:
zinc, silicon, fluorine and silver,
wherein the dental glass powder does not substantially contain aluminum.

2. A dental cement, comprising:
a first component containing the dental glass powder as claimed in claim 1; and
a second component containing a polycarboxylic acid polymer and water.
